(19) United States
(12) Patent Application Publication
(10) Pub. No.: US 2012/0289842 A1
(43) Pub. Date: Nov. 15, 2012

(54) NOVELTY GENDER PREDICTOR APPARATUS

(76) Inventor: Paul Gutman, Encino, CA (US)

(21) Appl. No.: 13/107,889

(22) Filed: May 14, 2011

(51) Int. Cl. A61B 5/02

(52) U.S. Cl. 600/502

(57) ABSTRACT

A portable and compact gender predictor apparatus intended as a novelty device comprised of a heart beat monitor, an indicator for gender, and a sensor, which predicts the gender of an unborn baby based on the heart beat rate of an unborn baby. In the preferred embodiment, the sensor detects the heart beat rate of an unborn baby when placed on the abdomen of a pregnant mother, and the heart beat monitor displays the heart beat of the unborn baby. When the heart beat rate of the unborn baby is 140 BPMs or higher, an indicator for the female gender is activated, and when the heart beat rate of the unborn baby is less than 140 BPMs, an indicator for the male gender is activated.

### CROSS-REFERENCE TO RELATED APPLICATION

None

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present invention generally relates to a novelty gender prediction apparatus purporting to predict the gender of a pregnant mother's baby.

Gender predictor apparatus are known. For example, U.S. Patent Application Publication No. US 2006/0110697 A1 (2004) by Taffinder teaches a baby gender message candle, namely, a device for containing, transforming or otherwise communicating a message intended to be a talisman or predictor of the gender of a pregnant mother's baby. A composite candle is built containing a message predictive of the gender of a pregnant mother's child based upon information selected by a third party. The device comprises a first outer candle, at least one second inner candle wherein the second inner candle is not visible until a portion of the first outer candle is consumed. Once the first outer candle is consumed, the second candle reveals the gender of a baby.

Also known is U.S. Pat. No. 4,779,871 (1986) to Rasmussen for a game for foretelling particulars of a person. Rasmussen teaches a game and method for foretelling particulars such as characteristics and future circumstances of a person, wherein a plurality of indicators are placed by participants, with their vision temporarily impaired, on a display device having a representation of a person therein. The predicted particulars which the selected person will have are those represented by or written upon the indicator positioned closest to a designated location on the display device. In one preferred embodiment, the game and method are used to predict the gender represented by the indicator which has been placed closest to a designated location on the display device with a representation of a baby thereon, and the birth date of the baby is predicted as the birth date that was previously written on that same indicator.

U.S. Pat. No. 4,788,984 (1988) to Marsik is a related known device. Marsik teaches a method and a kit for use in conceiving a child of a desired gender. The kit contains all information, instructions, and apparatus that is necessary for predicting ovulation time and for determining the time periods which define that time during which intercourse results in an improved probability of conceiving a child of the desired gender. The kit also contains information regarding the effects of the sperm placement location and timing of orgasm on gender selection. The kit includes information and instructions relating to practicing the method. A mucus chart is provided for monitoring the state of mucus of the woman attempting to conceive a child. Tissues are also part of the kit and are used in obtaining samples of the mucus. The kit further includes temperature sensors and a temperature chart for keeping track of the basal body temperature of the woman. These temperature-related products assist the user in confirming the prediction of ovulation. For ease of access and selection, the kit products are contained in individual packets of various sizes. Each of the packets is preferably housed in a convenient and desirably shaped container.

The above-referenced inventions are all based around myths, old wives tales, chance, and superstition. However, while Taffinder predicts gender based on the burning of an outer candle, Rasmussen bases predicts gender on the chance placement of an indicator on a display device while a mother's vision is partially impaired, and Marsik predicts gender on the timing of intercourse and the location of sperm placement, the present invention predicts gender based on the heart beat rate of a baby in the womb. According to one old wive's tale, if the heart rate of an unborn baby was 140+ beats per minute (BPM) the baby would be born a girl, and if the heart rate of the unborn baby was less than 140 BPMs, the unborn baby would be born a boy.

Thus, the objective of the present invention is to determine the gender of an unborn baby based on the baby's heart rate while in the womb. The objective is achieved in the form of an apparatus comprising a compact and portable heart beat monitor for gauging BPMs with indicators for gender and a sensor detachably connected to the heart rate monitor. While baby heart rate monitors are known and allow mothers to hear the heart beat rate of a baby, even some displaying the heart beat rate, none are known to comprise indicators for gender based on a particular heat beat rate.

### SUMMARY

The invention comprises a portable and compact unit comprised of a heart beat monitor, an indicator for gender, and a sensor detachably connected to the heart beat monitor, wherein the sensor detects the heart beat rate of an unborn baby when placed on the abdomen of a pregnant mother, and the heart beat monitor displays the heart beat of the unborn baby. When the heart beat of the unborn baby is 140 BPMs or higher, an indicator for the female gender is activated. When the heart beat of the unborn baby is less than 140 BPMs, an indicator for the male gender is activated. Operating buttons on the heart beat monitor operates the heart beat monitor, and can operate other functions, such as the sensor, or a clock and radio. A bridge connects the heart beat monitor and the sensor, and houses wires or cords connected between the heart beat monitor and sensor.

### DRAWING

FIG. 1 shows a compact and portable unit comprised of a heart beat monitor, and and two indicators, one identifying a girl and one identifying a boy, and a sensor attached to the heart beat monitor.

### DESCRIPTION

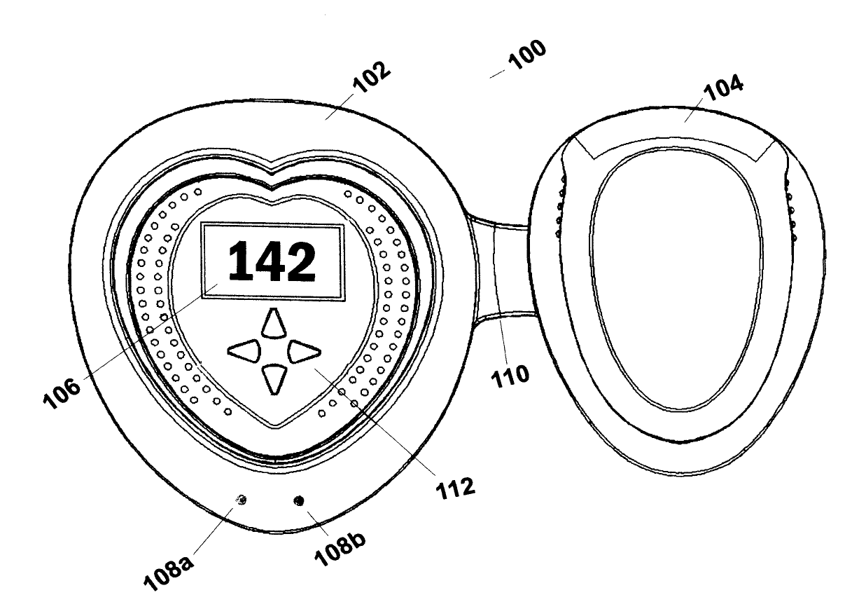

FIG. 1 shows the preferred embodiment of a gender predictor apparatus 100 comprised of a heart beat monitor 102 and a sensor 104. The heart beat monitor further comprises indicators 108 for gender, preferably one male 108a and one female 108b, operating buttons 112, and a display screen 106. The sensor 104 is detachably connected to the heart beat monitor, preferably by a bridge 110. The sensor 104 detects the heart rate of an unborn baby when placed on the abdomen of a pregnant mother. When the sensor detects the baby's heart beat, the beats per minute (BPMs) are displayed on the display screen 106. If the display screen registers 140 BPMs, or higher, an indicator 108 for the female 108b gender is activated. If the display screen registers less than 140 BPMs, an indicator for the male 108a gender is activated.

In another embodiment, the display screen 106 is used for other purposes, e.g., clock or radio station, while the indicators 108a, 108b alone convey the type of gender.

In an alternate embodiment, instead of two indicators for male and female, it is conceivable to have only one indicator 108 that is activated. For example, a male predictor apparatus can be activated only if the baby's heart rate is less than 140 BPMs, while a female predictor apparatus can only be activated if the baby's heart rate is 140 BPMs or greater.

In one preferred embodiment, the indicators are lights, which may be colored, for example, one pink light and one blue light. In another preferred embodiment, the indicators are sounds, which can comprise a sound message (e.g., “it's a boy,” “it's a girl”) or music.

The operating buttons 112 function to operate the display screen 106 (e.g., toggle between clock, radio, heart beats), adjust the indicators (e.g., volume, message), or to reset the heart beat monitor after each reading.

The bridge 110 allows the entire apparatus to be compact and portable, and treated as one unit. It is detachable so that the sensor 104 can be placed on a pregnant mother's abdomen. The sensor 104 can convey the heart beat rate to the heart beat monitor either through wires or cords (not shown) inside the bridge or wirelessly.

Although particular embodiments of the present invention have been illustrated in the accompanying drawing and described in the above description, it is to be understood that the present invention is not to be limited to just the embodiments disclosed, but that they are capable of numerous rearrangements, modifications and substitutions without departing from the description herein.

The following are claimed:

1. A gender predictor apparatus comprised of a heart beat monitor and a sensor characterized in that said monitor and sensor detects the heart beat of an unborn baby when the sensor is placed on the abdomen of a pregnant mother, said gender predictor apparatus predicts the gender of the unborn baby based on the heart beat rate of the unborn baby and conveys said gender by means of at least one indicator.

2. The gender predictor apparatus of claim 1, wherein the sensor is detachably connected to the heart beat monitor,
wherein the heart beat monitor comprises a display screen, at least one operating button, and the at least one indicator for gender, and
wherein the at least one indicator for gender identifies a female gender if the sensor detects a heart beat rate of 140 or more beats per minute, and a male gender if the sensor detects a heart beat rate of less than 140 beats per minute.

3. The gender predictor apparatus of claim 2, wherein the sensor is detachably connected to the heart beat monitor through a bridge.

4. The gender predictor apparatus of claim 3, wherein the bridge encloses wires or cords attached to the heart beat monitor and sensor.

5. The gender predictor apparatus of claim 2, wherein the display screen displays the heart beat rate of the unborn baby in beats per minute (BPMs).

6. The gender predictor apparatus of claim 1, wherein the at least one indicator for gender is in the form of at least one light or sound.

7. The gender predictor apparatus of claim 2, wherein the heart beat monitor further comprises a radio or clock.

8. The gender predictor apparatus of claim 7, wherein the at least one operating button operates the heart beat monitor or sensor.
